(19) United States
(12) Patent Application Publication
(10) Pub. No.: US 2026/0000917 A1
(43) Pub. Date: Jan. 1, 2026

(54) LOW-INTENSITY FOCUSED ULTRASOUND FOR TREATING POST-TRAUMATIC HEADACHE

(71) Applicant: The United States Government As Represented by The Department Of Veterans Affairs, Washington, DC (US)

(72) Inventors: John Coetzee, Palo Alto, CA (US); Byung Chul Yoon, Palo Alto, CA (US); Maheen M. Adamson, Palo Alto, CA (US)

(21) Appl. No.: 19/252,067

(22) Filed: Jun. 27, 2025

Related U.S. Application Data
(60) Provisional application No. 63/665,398, filed on Jun. 28, 2024.

(51) Int. Cl. A61N 7/00

(52) U.S. Cl. CPC A61N 7/00; A61N 2007/0021

(57) ABSTRACT

Method and system of treating post-traumatic headache using low-intensity focused ultrasound (LIFU).

### CROSS-REFERENCE TO RELATED APPLICATIONS

This application claims priority to U.S. Provisional Application No. 63/665,398, filed Jun. 28, 2024, the entirety of which is incorporated into this application by reference.

### BACKGROUND

Post-traumatic headache (PTH) is one of the most common sequelae of mild traumatic brain injuries (mTBI), accounting for 55-60% of post-concussion syndrome. Veterans are at high risk for PTH given their exposure to TBI from various situations including blasts, whiplash, falls, and vehicle crashes. In particular, migraine-like PTH has been reported to have a higher prevalence among persistent PTH and is associated with other co-morbidities including post-traumatic stress disorder (PTSD). The current mainstay treatment for PTH is pharmacologic, which can be limited due to side effects, non-efficacy, and non-compliance. For instance, an observational study of US soldiers on treatment outcomes of chronic PTH showed a 30-40% rate of medication discontinuation at 3 months of therapy. A need for improved therapies exists in the art.

### SUMMARY

Neuromodulation of the head of the caudate nuclei using low-intensity focused ultrasound (LIFU) can be a treatment for migraine-like PTH. The caudate nucleus is thought to be one of the pain mediators in the pathophysiology of migraine. Patients with more frequent migraine attacks (>15 attacks per month) had a significantly lower response on fMRI to pain in the basal ganglia including the caudate nuclei when compared to those with low frequency of migraine attacks. In addition, the caudate nuclei were larger in those with high-frequency migraine attacks, suggesting that there is both structural and functional change to the caudate that is associated with the severity of migraine symptoms.

The disclosed treatment method comprises determining locations of a first and a second caudate nucleus head inside the brain of the subject; and transmitting focused energy to the first and second caudate nucleus heads through an ultrasound probe of an ultrasound device under conditions sufficient to reduce the severity of one or more symptoms of post-traumatic headache in the subject.

### BRIEF DESCRIPTION OF THE DRAWINGS

The foregoing summary, as well as the following description of the disclosure, is better understood when read in conjunction with the appended drawings. For the purpose of illustrating the disclosure, the drawings illustrate some, but not all, alternative embodiments. This disclosure is not limited to the precise arrangements and instrumentalities shown. The following figures, which are incorporated into and constitute part of the specification, assist in explaining the principles of the disclosure.

FIG. 1 shows 3D-volumetric T1-weighted images used for localization and low-intensity focused ultrasound (LIFU) neuromodulation. Circles denote LIFU entry sites, with circles on cross-hairs denoting the heads of the caudate nuclei bilaterally, and purple lines connecting both circles denote LIFU trajectories. The bottom right panel shows surface rendering with the skin entry site (dot).

FIG. 2A shows a plot of pain severity scores at baseline and during LIFU treatment for Participant A (0=no pain, 10=most severe pain). Participant A showed more fluctuating pain score changes relative to Participant B (FIG. 2B).

FIG. 2B shows a plot of pain severity scores at baseline and during LIFU treatment for Participant B (0=no pain, 10=most severe pain). The results from Participant B showed a more substantial decrease in pain severity scores relative to Participant A (FIG. 2A).

FIG. 3A shows a plot of pain interference score (0=no interference, 10=most interference) with various functions for Participant A.

FIG. 3B shows a plot of pain interference score (0=no interference, 10=most interference) with various functions for Participant B. Participant B showed a more substantial decrease in pain interfering with function relative to Participant A.

FIG. 4A shows a plot of pain score reflective of headache symptoms which were tracked before, during, and after LIFU treatments for Participant A.

FIG. 4B shows a plot of pain score reflective of headache symptoms which were tracked before, during, and after LIFU treatments for Participant B.

FIG. 5 shows a schematic of an overview of an exemplary treatment procedure in which a subject undergoes neuronavigated sonication on five different days.

FIG. 6 shows a system which includes an MRI machine, ultrasound probe, a processing unit, and surgical navigation system for performing the described LIFU treatments.

### DETAILED DESCRIPTION

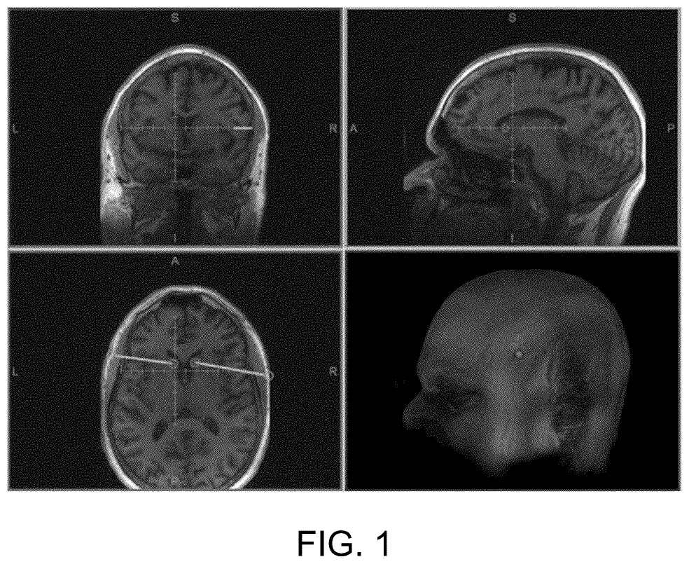

Prior to treatment, a subject may undergo an imaging analysis to determine locations of a first and a second caudate nucleus head inside the brain of the subject. Imaging may comprise magnetic resonance imaging (MRI). The caudate nucleus is a paired, “C”-shaped subcortical structure which lies inside the brain near the thalamus. It plays a role in higher neurological functions. Each caudate nucleus includes a large anterior head, a body, and a thin tail that wraps anteriorly such that the caudate nucleus head and tail can be visible in the same coronal cut. FIG. 1 (lower left quadrant) shows bilateral caudate nuclei heads in the interior of a subject's brain tissue. These heads are target treatment regions for delivering focused energy such as low-intensity focused ultrasound (LIFU) energy to the subject.

Focused energy is transmitted to the first and second caudate nucleus heads through an ultrasound probe of an ultrasound device under conditions sufficient to reduce the severity of one or more symptoms of post-traumatic headache in the subject. During treatment, the target caudate nuclei heads can be targeted outside an ultrasound scanner using a neuronavigation system such as LOCALITY or BRAINSIGHT. The ultrasound energy can be administered inside an MRI machine in some embodiments, using an MR safe transducer such as one manufactured by Brainsonix. The ultrasound transducer probe can be configured to transmit low-intensity focused ultrasound (LIFU). This can be accomplished for instance at a frequency of 500 to 650 KHz. Depending on the size of the subject's head, focused energy can be transmitted to the first and second caudate nucleus heads at a sonication depth of 40 to 80 mm. With an ultrasound device such as a Brainsonix device, this can be achieved by using different transducers with different fixed focal lengths.

In some embodiments, the subject may undergo 2-10 daily treatment sessions. Each session may include a 5-40 minute sonication of a first caudate nucleus head of the subject, followed by a 5-40 minute sonication of a second caudate nucleus head of the subject. In some embodiments, the treatment method can be repeated 2-10 times per day. It can be preferable in other embodiments to have no more than one session per day, or no more than 1-10 sessions per day. In some embodiments, treatment sessions can occur on consecutive days.

Treatment can be administered using a suitable ultrasound device with a transducer probe, such as a Brainsonix single element transducer, or a similar device. In some embodiments, the ultrasound device can have a single transducer, be capable of producing the relevant sonication pattern, have built in limits to prevent exceeding safety thresholds, and if desired, magnetic resonance compatibility. The transducer probe can be aimed by hand, using head straps for example, or using an adjustable mechanical or robotic arm into which the device can be placed. Between the patient's head and the device can preferably be ultrasound gel or mineral oil. A subject's hair does not need to be shaved, but it is preferable.

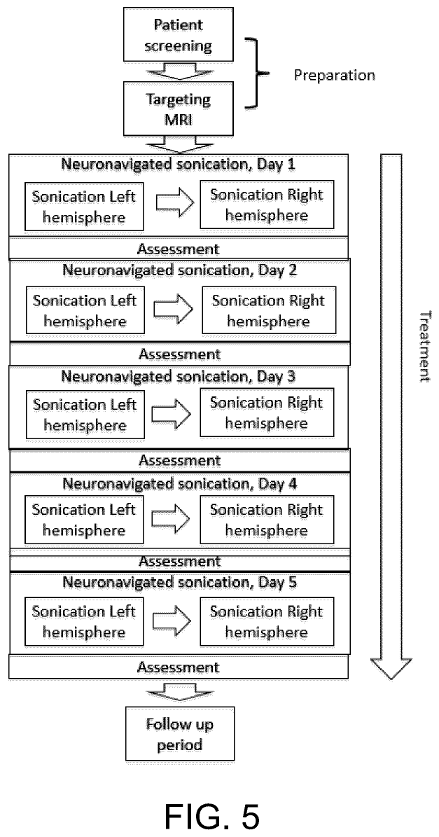

An exemplary treatment protocol is shown in FIG. 5. According to this embodiment, focused energy is delivered consecutively to each caudate head using neuronavigation assisted sonication, followed by an assessment. This treatment sequence is repeated for five days or longer (e.g., from 5 to 30 days or from 5 to 15 days) with assessments being conducted periodically to determine whether severity of any symptom of post-traumatic headache has been reduced.

In some non-limiting embodiments, the following sonication parameters can be used: Frequency: 500-650 KHz; Pr.0 (MPa) (specific acoustic pressure amplitude): 0.190-0.800; Pr.3 (MPa) (specific acoustic pressure amplitude): 0.170-0.650; ISPTA.0 (mW/cm²) (Spatial-Peak Temporal-Average Intensity): 600-1000; ISPTA.3 (mW/cm²) (Spatial-Peak Temporal-Average Intensity): 500-900; XMT volts: 2-10; Pulse width: 1-10; Pulse repetition frequency (PRF, in Hz): 10-250; Duty cycle (percentage of time the ultrasonic power is on during each pulse): 0.1-0.8; Sonication number (the number of sonication cycles): 8-12; Duration(s) (duration of each sonication cycle): 10-40; Between(s) (time between each sonication cycle): 10-40.

In one aspect, the sonication parameter can comprise a frequency of 500-650 KHz. In a further aspect, the sonication parameter can include a Pr.0 (MPa): 0.190-0.800. In a further aspect, the sonication parameter can include a Pr.3 (MPa): 0.170-0.650. In a further aspect, the sonication parameter can include a ISPTA.0 (mW/cm²): 600-1000. In a further aspect, the sonication parameter can include a XMT volts: 2-10. In a further aspect, the sonication parameter can include a Pulse width: 1-10. In a further aspect, the sonication parameter can include a Pulse repetition frequency (PRF, in Hz): 10-250. In a further aspect, the sonication parameter can include a Duty cycle: 0.1-0.8. In a further aspect, the sonication parameter can include a Sonication number: 8-12. In a further aspect, the sonication parameter can include a Duration(s): 10-40; Between(s): 10-40. In one aspect, the sonication parameters include one or more or all of the foregoing sonication parameters. These parameters are understood by those skilled in the art and may be used with a Brainsonix device for instance.

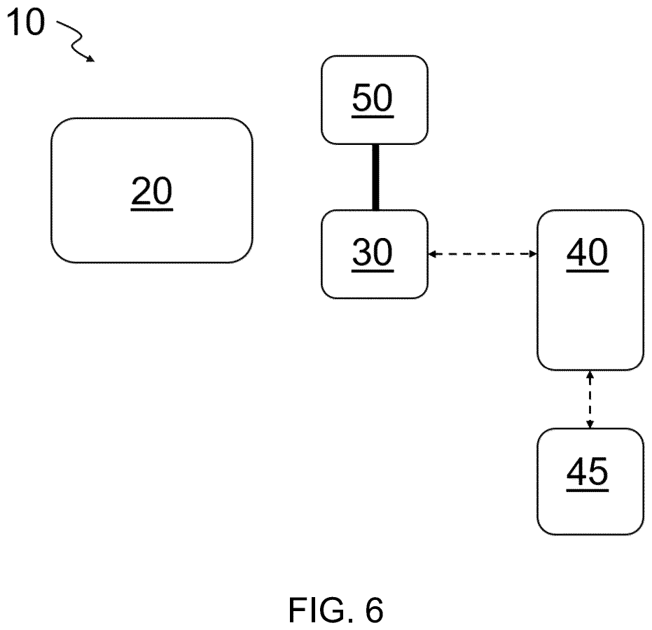

In one embodiment, referring to FIG. 6, a system 10 can include an MRI machine 20 and an ultrasound probe 30. The ultrasound probe 30 can in one embodiment be coupled to a processing unit 40 and a surgical navigation system 45, such that the processing unit 40 can receive data from the navigation system 45 that is indicative of locations of respective caudate nucleus heads. The processing unit 40 can then determine preferred locations or orientations that will allow the ultrasound probe 30 to provide LIFU to the caudate nuclei. In one embodiment, the location of the ultrasound probe 30 can be controlled with a robotic arm 50 such that precise placement of the probe may be accomplished in an automated fashion (for example, via the processing unit 40 directing movement of the robotic arm 50 based on the determined locations of the caudate nucleus heads). Optionally, the processing unit 40 can be in communication with a display that displays data or images from the MRI machine and/or the surgical navigation system. Optionally, the processing unit 40 and/or the display can be provided as components of a computing device, such as a computer, a tablet, or a smartphone.

The described method makes it possible to administer an effective treatment to individuals with treatment resistant chronic headache who have been unresponsive to other treatment modalities. By enabling the noninvasive targeting of brain structures that give rise to headache in a safe and tolerable manner, it is possible to give individuals who suffer from these headaches (including many Veterans) a chance at having a more rewarding and productive life.

### EXAMPLES

The following examples further illustrate this disclosure. The scope of the disclosure and claims is not limited by the scope of the following examples.

Two Veterans with a history of mTBI and persistent, migraine-type PTH underwent daily LIFU treatment for 5 consecutive days in this study approved by the Institution Review Board and VA Research and Development Review Board. The first LIFU session was performed within a 3T MRI scanner (GE Premiere) where Brain MRI was performed before, during, and after the LIFU administration (Brainsonix BX Pulsar 1002). The LIFU parameter was 650 kHz frequency, 5-millisecond pulse width, 10 Hz pulse repetition frequency, 30-second duration, and 30-second interval between pulses. Baseline MRI included diffusion weighted imaging, susceptibility weighted imaging, diffusion tensor imaging, T2/FLAIR, T2, and 3D MP-RAGE sequences. Arterial spin labeling (ASL) was also obtained given the prior findings that LIFU with comparable parameters to our study resulted in a reduction of cerebral perfusion on ASL. During the first session, LIFU was targeted to the left caudate head using a real-time T1 weighted sequence; only the left side was targeted due to the limited MRI scan time. Subsequent sessions 2 to 5 were performed in the clinic with LIFU targeted to bilateral caudate heads using the same LIFU parameters. The localization was performed using Localite neuronavigation system loaded with each patient's MP-RAGE sequence. Session #5 was followed by a post-treatment MRI with an identical protocol as the baseline MRI. Each patient also had a 1-month follow-up visit.

Patient B demonstrated substantial improvement in headache severity as assessed by the Brief Pain Inventory. For instance, the worst pain within 24 hours decreased from 9 (0=no pain, 10=most severe pain) at baseline to 2 following the last LIFU session. Patient A showed less pronounced change during the LIFU sessions with the worst pain score decreasing from 9 at baseline to 7. Similarly, patient B had substantial improvement in the degree to which headache interferes with various daily functions such as mood, mobility, sleep, work, etc. while patient A demonstrated generally improving but more modest change. At the 1-month follow-up visit, both participants reported that the headache symptoms had returned to baseline. Functional impairment from headache improved for patient B as demonstrated by the improved Migraine Disability Assessment (MIDAS) score, which decreased by 54% from 81 at baseline to 49 post-LIFU. Patient A did not demonstrate any notable improvement with the baseline and post-LIFU MIDAS scores of 64 and 69, respectively. Both patients had co-morbid PTSD, and patient B showed improved PTSD symptoms as assessed by the PTSD checklist for DSM-5 (PCL-5) with the baseline score of 53 and post-LIFU score of 34. Participant A showed no change. Both participants demonstrated no substantial change in cognitive assessment using MoCA. On ASL, Patient B showed a decreasing trend in the normalized cerebral blood flow in the left caudate head only (0.81 baseline, 0.73 post-LIFU session 1, 0.48 post-LIFU session 5. The right side did not demonstrate any notable trend (0.76, 0.53, 0.70). Patient A also did not demonstrate any notable change (Left: 0.93, 1.08, 1.07; Right: 0.72, 0.94, 0.78). No moderate to severe adverse effects were observed. Patient B reported a mild, transient headache during LIFU, which was not worse than baseline and resolved after each session. Patient A did not report any adverse effects. None of the participants showed acute intracranial abnormalities such as infarction, hemorrhage, and edema on MRI.

Each participant underwent five daily LIFU treatments as follows. With reference to FIG. 1, images were collected on day 1 with left caudate head targeting with an MRI scanner. On days 2-5, bilateral caudate head targeting was conducted in the clinic using localized neuronavigation. A BrainSonix BX Pulsar 1002 LIFU system was used. Multiple MRIs were obtained before LIFU treatments, during the first LIFU session, and after the last LIFU session. Each participant also underwent cognitive, symptomatic, and/or behavioral assessments before, during, immediately after, and 1 month after the LIFU treatments.

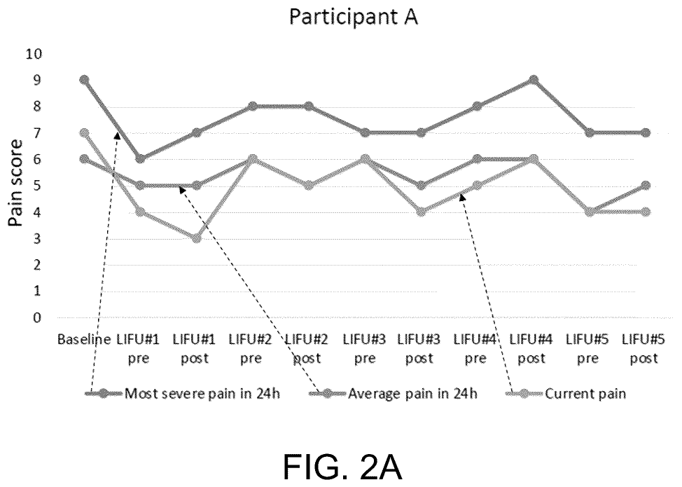

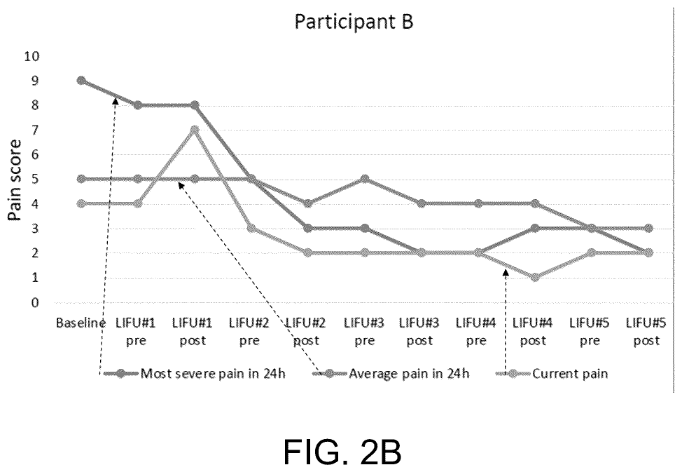

With reference to FIGS. 2A-2B, pain severity scores at baseline and during LIFU treatment (0=no pain, 10=most severe pain) demonstrate a more substantial decrease in the pain severity scores for Participant B. Participant A showed more fluctuating pain score changes. No substantial changes in pain scores were noted before and after each LIFU session.

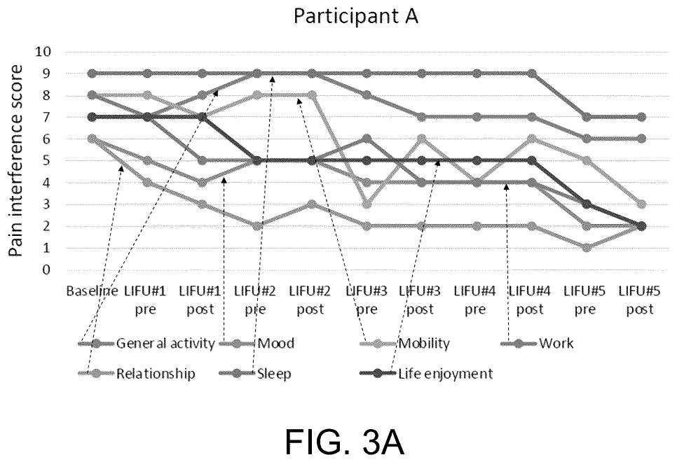

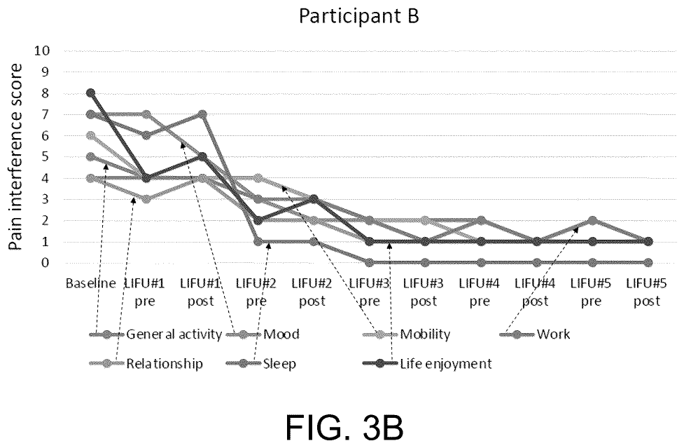

With reference to FIGS. 3A-3B, degrees to which pain interfere with various aspects of daily function (0=no interference, 10=most interference) were also assessed. Participant B showed a more substantial decrease in pain interfering with function.

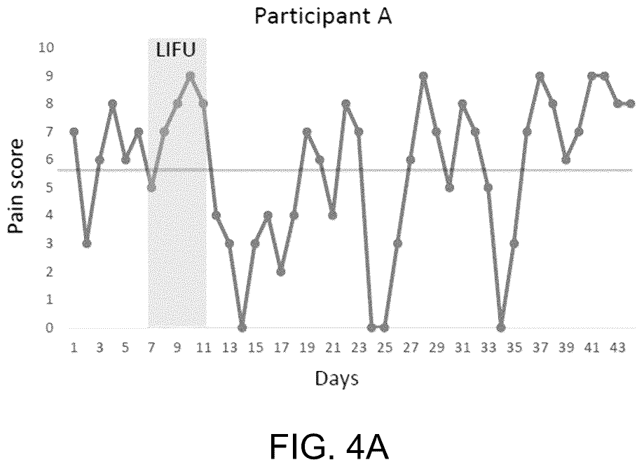

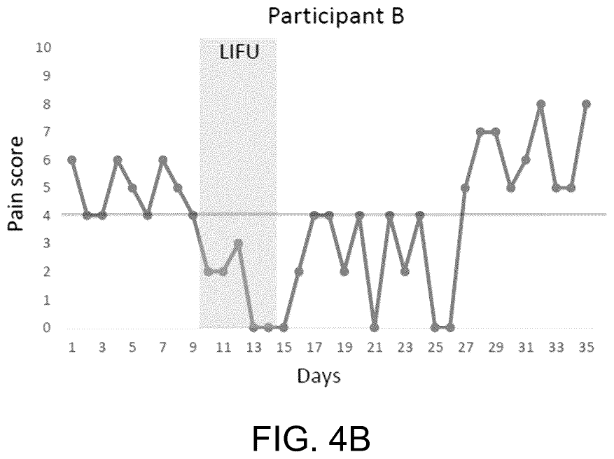

Referring to FIGS. 4A-4B, headache symptoms were tracked before, during, and after LIFU treatments. Both participants demonstrated improvements in symptoms following LIFU, compared to baseline. However, both participants reported that headache symptoms were back to baseline at the 1-month follow-up. The horizontal line denotes the mean pain score for each participant.

Additional behavioral and cognitive tests were performed, results of which are shown in Table 1. MIDAS=Migraine Disability Assessment Test (Scores>21=Grade IV=Severe Disability). Note: MIDAS assesses symptoms in the past 3 months. PCL-5=Post traumatic stress disorder (PTSD) checklist for DSM-5. MoCA=Montreal Cognitive Assessment

TABLE 1
Behavioral and Cognitive Assessments

| | Participant A | | Participant B | |
|---|---|---|---|---|
| | Pre-LIFU | Post-LIFU#5 | Pre-LIFU | Post-LIFU#5 |
| MIDAS | 64 | 68 | 81 | 49 |
| PCL-5 | 64 | 64 | 53 | 34 |
| MoCA | 30 | 29 | 27 | 29 |

Caudate head targeting was more challenging when using MRI only, compared to Localite neuronavigation due to the limited space within the MRI scanner/head coil and the need for repeat imaging after each transducer adjustment. Participant A reported mild head during LIFU session that was transient and was not worse than the baseline headache. Participant B reported no adverse effects. Participant B also showed more substantial improvements in symptoms. This study demonstrates that the LIFU targeting of bilateral caudate heads is feasible for the treatment of post-traumatic headache (PTH).

Features and advantages of this disclosure are apparent from the detailed specification, and the claims cover all such features and advantages. Numerous variations will occur to those skilled in the art, and any variations equivalent to those described in this disclosure fall within the scope of this disclosure. Those skilled in the art will appreciate that the conception upon which this disclosure is based may be used as a basis for designing other compositions and methods for carrying out the several purposes of this disclosure. As a result, the claims should not be considered as limited by the description or examples.

What is claimed is:

1. A method of treating post-traumatic headache in a subject, comprising:
determining locations of a first and a second caudate nucleus head inside the brain of the subject;
transmitting focused energy to the first and second caudate nucleus heads through an ultrasound probe of an ultrasound device under conditions sufficient to reduce the severity of one or more symptoms of post-traumatic headache in the subject.

2. The method of claim 1, wherein focused energy comprises low-intensity focused ultrasound (LIFU).

3. The method of claim 1, wherein focused energy is transmitted to the first and second caudate nucleus heads using a neuronavigation system.

4. The method of claim 1, wherein focused energy is transmitted to the first and second caudate nucleus heads at a frequency of 500 to 650 KHz.

5. The method of claim 1, wherein focused energy is transmitted to the first and second caudate nucleus heads at a sonication depth of 40 to 80 mm.

6. The method of claim 1, wherein focused energy is transmitted to the first caudate nucleus head for a time of 5 to 40 minutes, followed by focused energy being transmitted to the second caudate nucleus head for a time of 5 to 40 minutes.

7. The method of claim 1, wherein the treatment method is repeated 2-10 times per day.

8. The method of claim 1, wherein the locations of the first and second caudate nucleus heads are determined by magnetic resonance imaging (MRI).

9. The method of claim 1, wherein the subject's post-traumatic headache is a result of traumatic brain injury.

10. The method of claim 1, wherein the subject has treatment-resistant post-traumatic headache.

11. A system for treating post-traumatic headache in accordance with the method of claim 1.
